(19) United States
(12) Patent Application Publication
(10) Pub. No.: US 2026/0033815 A1
(43) Pub. Date: Feb. 5, 2026

(54) BIOPSY FORCEPS, ENDOSCOPIC TREATMENT SYSTEM, AND METHOD FOR COLLECTING TISSUE USING ENDOSCOPIC TREATMENT SYSTEM

(71) Applicants: National Cancer Center, Tokyo (JP); OLYMPUS CORPORATION, Tokyo (JP)

(72) Inventors: Tatsunori TSUNETO, Hino-shi (JP); Yuji MATSUMOTO, Tokyo (JP)

(21) Appl. No.: 19/351,906

(22) Filed: Oct. 7, 2025

Related U.S. Application Data
(63) Continuation of application No. PCT/JP2023/014507, filed on Apr. 10, 2023.

(51) Int. Cl. A61B 10/06; A61B 1/267; A61B 10/04; A61B 17/34

(52) U.S. Cl. CPC A61B 10/06; A61B 1/2676; A61B 10/04; A61B 17/3415

(57) ABSTRACT

The present disclosure is a biopsy forceps that is insertable into a puncture hole formed by puncturing biological tissue with a needle, including a first forceps piece having a length in one direction and a forceps cup having a recess in which the biological tissue is collectable, and a second forceps piece disposed on an opening side of the recess of the first forceps piece, wherein the first forceps piece is openable and closeable toward a distal end side in a longitudinal direction with respect to the second forceps piece, the forceps cup has a body portion and a distal end portion provided on a distal end side of the body portion in the longitudinal direction, and a length of the distal end portion in a direction perpendicular to the longitudinal direction becomes smaller toward the distal end side.

### CROSS-REFERENCE TO RELATED APPLICATIONS

This application is a continuation application based on PCT Patent Application No. PCT/JP2023/014507, filed on Apr. 10, 2023, the entire content of which is hereby incorporated by reference.

### FIELD

The present disclosure relates to a biopsy forceps, an endoscopic treatment system, and a method for collecting tissue using the endoscopic treatment system.

### BACKGROUND

Conventionally, endobronchial ultrasound-guided transbronchial needle aspiration (EBUS-TBNA) in which an ultrasound endoscope and aspiration biopsy needle are used to puncture the bronchial wall and collect biological tissue from lymph nodes to be examined has been commonly used as a diagnostic method for lung cancer. Although the spread of EBUS-TBNA has significantly improved the accuracy of lung cancer diagnoses, there is a need to further improve the quantity and quality of samples that can be collected by needle aspiration biopsy. In addition, since blood is also aspirated, it is desirable to collect tissue without mixing the tissue with blood in order not to affect an accurate diagnosis rate.

As a solution to the above problem, a technique has been reported in which a biopsy forceps is inserted into a site (hereinafter, referred to as a puncture hole) in which tissue is punctured with a needle during EBUS-TBNA to collect the tissue of lymph nodes (for example, Japanese Unexamined Patent Application, First Publication No. 2000-201939, and Japanese Unexamined Patent Application, First Publication No. 2003-290229).

According to the report, major benefits expected are that there is less risk of crushing a sample or blood contamination, and that a sufficient amount of sample can be collected to diagnose lung cancer.

### SUMMARY

The present disclosure provides a biopsy forceps that has excellent insertability into a puncture hole and can ensure a sufficient collection amount of tissue, an endoscopic treatment system, and a method for collecting tissue using the endoscopic treatment system.

A biopsy forceps according to a first aspect of the present disclosure is a biopsy forceps that is insertable into a puncture hole formed by puncturing biological tissue with a needle, including a first forceps piece having a length in one direction and a forceps cup having a recess in which the biological tissue is collectable, and a second forceps piece disposed on an opening side of the recess of the first forceps piece, wherein the first forceps piece is openable and closeable toward a distal end side in a longitudinal direction with respect to the second forceps piece, the forceps cup has a body portion and a distal end portion provided on a distal end side of the body portion in the longitudinal direction, and a length of the distal end portion in a direction perpendicular to the longitudinal direction becomes smaller toward the distal end side.

A endoscopic treatment system according to the first aspect of the present disclosure includes an endoscope insertable into a bronchus, and the biopsy forceps.

A method for collecting tissue using the endoscopic treatment system according to a first embodiment of the present disclosure includes inserting a biopsy forceps in a closed state into an inside of a lumen, and advancing the biopsy forceps to a vicinity of a puncture hole formed by puncturing a puncture needle from the inside of the lumen into a lumen wall of the lumen and biological tissue, inserting the biopsy forceps into the puncture hole from a distal end side, and opening and closing the biopsy forceps inside the biological tissue to collect a part of the biological tissue with the biopsy forceps.

### BRIEF DESCRIPTION OF DRAWINGS

FIG. 1 is a diagram showing an overall configuration of an endoscopic treatment system including a biopsy forceps according to an embodiment.

FIG. 2 is an overall view showing the biopsy forceps.

FIG. 3 is a cross-sectional view of a sheath taken along line III-III shown in FIG. 2.

FIG. 4 is a perspective view showing a configuration of a support member and a forceps in a closed state.

FIG. 5 is a perspective view showing a configuration of the support member and the forceps in an open state.

FIG. 6 is an exploded perspective view showing the configuration of the support member and the forceps.

FIG. 7 is a plan view showing a configuration of a forceps piece (a first member).

FIG. 8 is a cross-sectional view taken along line VIII-VIII in FIG. 7.

FIG. 9 is a cross-sectional view taken along line VX-VX in FIG. 7.

FIG. 10 is a cross-sectional view taken along line X-X in FIG. 7.

FIG. 11 is a perspective view showing the configuration of the forceps piece.

FIG. 12 is a flow diagram showing operation steps in the endoscopic treatment system.

FIG. 13 is a perspective view showing a forceps piece of a second embodiment.

FIG. 14 is a perspective view showing a forceps piece of a third embodiment.

FIG. 15 is a perspective view showing a forceps in a closed state with a forceps piece of a fourth embodiment.

FIG. 16 is a perspective view showing the forceps in an open state with the forceps piece of the fourth embodiment.

FIG. 17 is a perspective view showing a forceps piece of the fourth embodiment.

FIG. 18 is a perspective view showing a forceps piece of a fifth embodiment.

FIG. 19 is a perspective view showing the forceps piece of the fifth embodiment.

### DETAILED DESCRIPTION

Conventional biopsy forceps have excellent insertability into a puncture hole, but a cup capacity is small, and thus it is difficult to ensure a sufficient collection amount of tissue. In contrast, when a biopsy forceps having a larger cup capacity than the above biopsy forceps is used, a sufficient collection amount of tissue can be ensured, but there is a problem that the insertability into the puncture hole is poor.

The present disclosure can provide a biopsy forceps that can ensure a sufficient collection amount of tissue and has excellent insertability into a puncture hole. The present disclosure can provide an endoscopic treatment system capable of identifying and collecting target tissue (lesion tissue) within a bronchus using the biopsy forceps and an endoscope. The present disclosure can provide a method for collecting tissue using the endoscopic treatment system that can reduce a burden of a collecting operation since a sufficient amount of sample required for biopsy can be collected using the biopsy forceps.

### First Embodiment

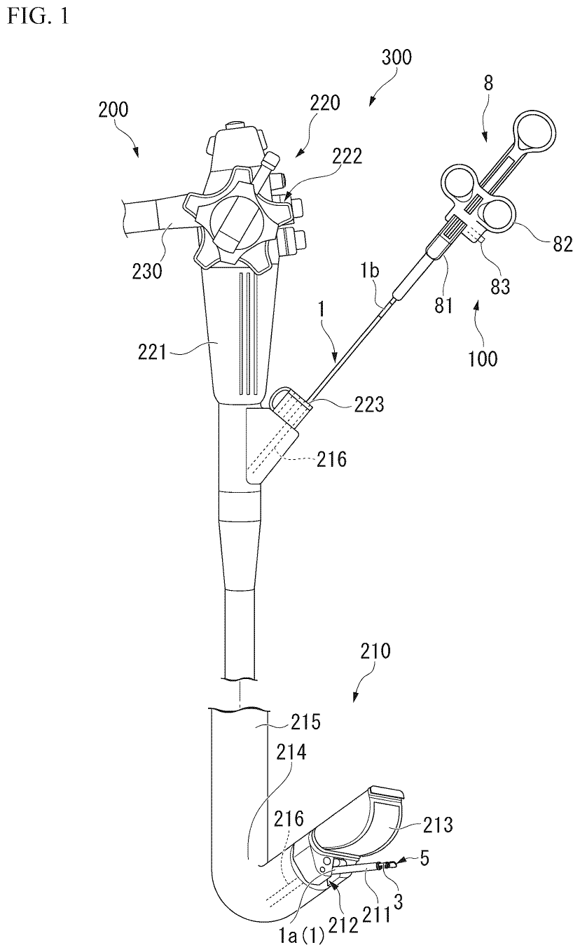

A first embodiment of the present disclosure will be described with reference to FIGS. 1 to 11.

In the following drawings, dimensions of components may be at different scales to make it easier to see each of the components.

FIG. 1 is a diagram showing an overall configuration of an endoscopic treatment system 300 including a biopsy forceps 100 according to this embodiment.

### [Endoscopic Treatment System 300]

The endoscopic treatment system 300 is used for biopsies to collect tissue samples to diagnose lung cancer. As shown in FIG. 1, the endoscopic treatment system 300 includes the biopsy forceps 100 according to the present disclosure, a known puncture needle (not shown), and a known ultrasonic endoscope (an endoscope) 200. The biopsy forceps 100 and the puncture needle are used by alternately inserting one each into the ultrasonic endoscope 200. In the endoscopic treatment system 300, the biopsy forceps 100 and the puncture needle may be appropriately replaceable with respect to the ultrasonic endoscope 200.

### [Ultrasound Endoscope 200]

The ultrasound endoscope 200 is a known flexible endoscope, and includes an insertion part 210 that is inserted into the body from the distal end, an operation part 220 installed at a proximal end of the insertion part 210, and a universal cord 230 installed at the operation part 220.

The insertion part 210 is a long, thin member that may be insertable into a lumen. The insertion part 210 has a distal end portion 211, a bending portion 214, and a flexible portion 215. The distal end portion 211, the bending portion 214, and the flexible portion 215 are connected in this order from the distal end side. A channel 216 for inserting the biopsy forceps 100 is provided inside the insertion part 210. A distal end opening 212 of the channel 216 and an imaging part (a ultrasonic transmission/reception part) 213 are provided in the distal end portion 211.

The imaging part 213 includes an ultrasonic transducer array such as a PZT or CMAT, and is capable of acoustically imaging an area to be treated. The bending portion 214 bends in accordance with a user's operation of the operation part 220. The flexible portion 215 is a tubular portion having flexibility.

The operation part 220 is connected to the flexible portion 215. The operation part 220 has a grip 221, an input part 222, and a forceps port 223. The grip 221 is a member that is supported by a user. The input part 222 receives an operation input for bending the bending portion 214. The forceps port 223 is a proximal end opening of the channel 216.

The universal cord 230 connects the ultrasonic endoscope 200 to an external device. An imaging cable or the like that outputs an imaging signal captured by the imaging part 213 to the outside is inserted into the universal cord 230.

### [Biopsy Forceps 100]

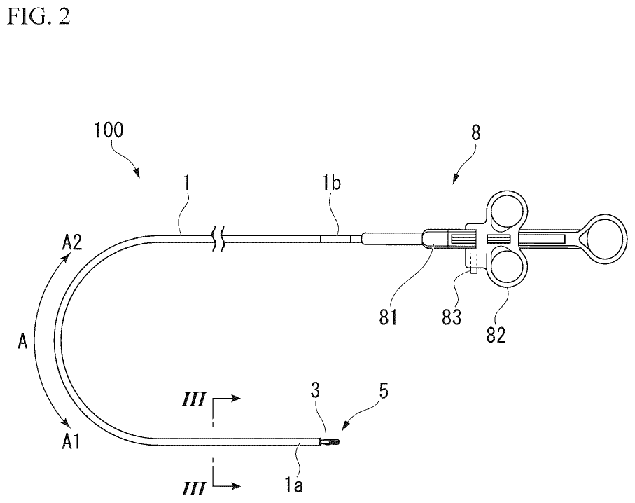

FIG. 2 is an overall view showing the biopsy forceps 100.

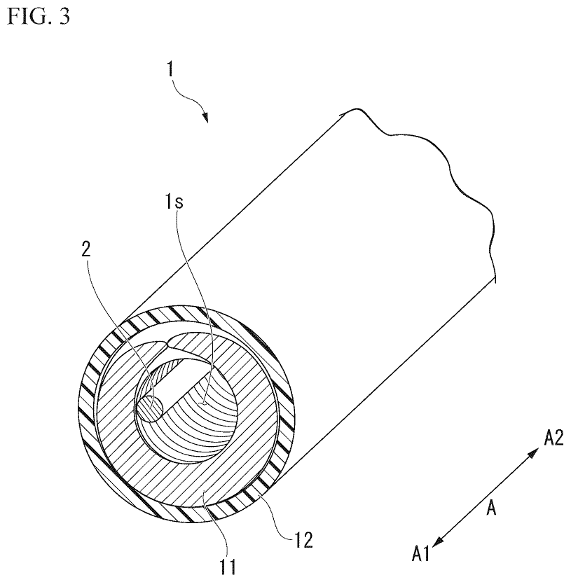

The biopsy forceps 100 includes a sheath 1, an operation wire 2 (refer to FIG. 3), an operating part 8, a support member 3, and a forceps (a jaw) 5.

In the following description, as shown in FIG. 2, in a longitudinal direction A along a central axis of the biopsy forceps 100, the side inserted into a patient's body is referred to as the “distal end side A1” and the side of the operating part 8 is referred to as the “proximal end side A2.”

The support member 3 and the forceps 5 are provided at the distal end of the biopsy forceps 100. The forceps 5 are supported by the support member 3 to be openable and closable. As shown in FIGS. 1 and 2, the support member 3 has the proximal end side A2 installed at a distal end 1a of the sheath 1, and supports the forceps 5 at the distal end side A1.

### [Sheath 1]

The sheath 1 is a flexible and long member that extends from the distal end 1a to the proximal end 1b as shown in FIGS. 1 and 2. The sheath 1 has an outer diameter that allows the sheath 1 to be inserted into the channel 216 of the ultrasonic endoscope 200. As shown in FIG. 1, in a state in which the sheath 1 is inserted into the channel 216, the distal end 1a of the sheath 1 may protrude and retract from the distal end opening 212 of the channel 216. The sheath 1 may have insulating properties.

FIG. 3 is a cross-sectional view of the sheath 1 taken along line III-III shown in FIG. 2.

The sheath 1 includes a sheath 11 formed by spirally winding a metal wire. A tube sheath made of a resin is provided in an internal space Is of the sheath 1. The operation wire 2 is inserted through the tube sheath. This configuration can reduce friction between the operation wire 2 and the sheath 1. However, this embodiment is not limited thereto, and a covering tube 12 that covers an outer circumferential surface of the sheath 11 may be provided.

### [Operation Wire 2]

The operation wire 2 is a metal wire that is inserted through the internal space 1s of the sheath 1 shown in FIG. 3. As shown in FIGS. 1 and 2, the distal end of the operation wire 2 is connected to the forceps 5, and the proximal end of the operation wire 2 is connected to the operating part 8. As shown in FIG. 3, the operation wire 2 is inserted through the internal space Is of the sheath 1.

In the following description, a direction in which the forceps 5 opens and closes is referred to as an “opening-closing direction B” or an “up-down direction B.” Moreover, a direction perpendicular to the longitudinal direction A and the opening-closing direction B is defined as a “width direction C” or a “left-right direction C.” Moreover, a surface parallel to the longitudinal direction A and the width direction Cis defined as a “grip surface GP.”

In the following description, a direction in which a forceps piece (a first forceps piece) 6 opens is referred to as the “lower side B1” in the opening-closing direction B, and a direction in which a forceps piece (a second forceps piece) 7 opens is referred to as the “upper side B2” in the opening-closing direction B. In addition, a direction facing right when seen from the distal end side A1 to the proximal end side A2 is referred to as the “right side C1” in the width direction C, and a direction facing left is referred to as the “left side C2” in the width direction C.

### [Operating Part 8]

As shown in FIGS. 1 and 2, the operating part 8 is provided on the proximal end side A2 of the sheath 1. The operating part 8 includes an operating part main body 81, a slider 82, and a power supply connector 83.

A distal end portion of the operating part main body 81 is connected to the proximal end 1b of the sheath 1. The operating part main body 81 has an internal space through which the operation wire 2 may be insertable. The operation wire 2 passes through the internal space of the sheath 1 and the internal space of the operating part main body 81 and extends to the slider 82.

The slider 82 is installed on the operating part main body 81 to be movable in the longitudinal direction A. The proximal end of the operation wire 2 is connected to the slider 82. The operation wire 2 advances and retracts when the user advances and retracts the slider 82 relative to the operating part main body 81.

In addition, when the user rotates the operating part 8, the slider 82 rotates the operation wire 2, and accordingly the forceps 5 rotates.

The power supply connector 83 is fixed to the slider 82.

The power supply connector 83 can be connected to a high-frequency power supply device (not shown), and is electrically and physically connected to the proximal end of the manipulation wire 2. The power supply connector 83 is capable of supplying high-frequency current, which is supplied from a high-frequency power supply device, to the forceps 5 via the operation wire 2 and the support member 3.

The power supply connector 83 may not be provided.

### [Support Member 3]

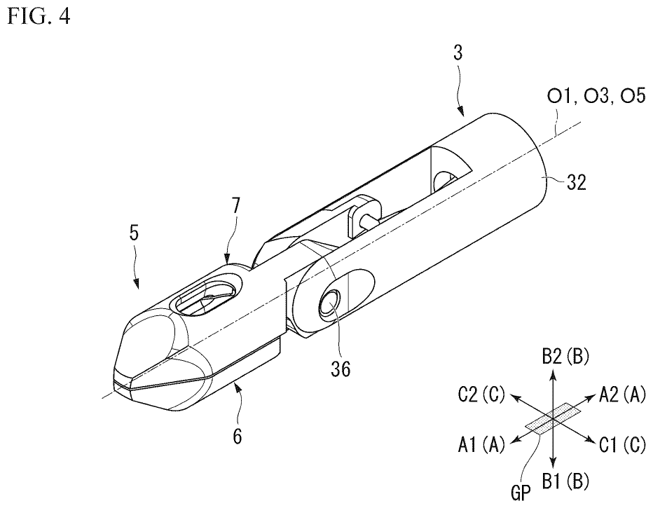

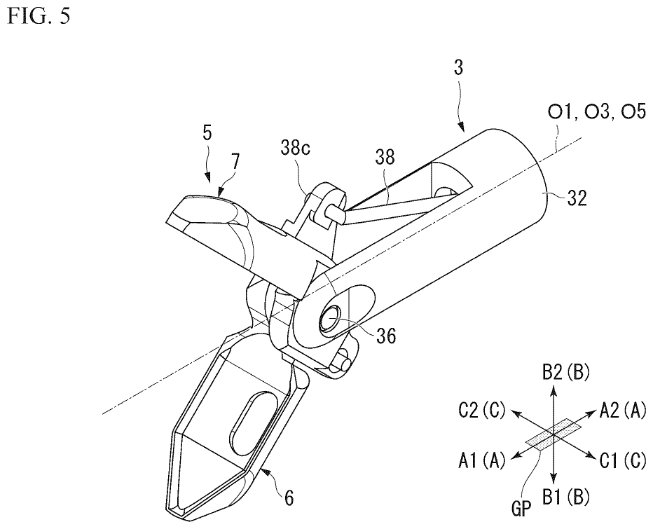

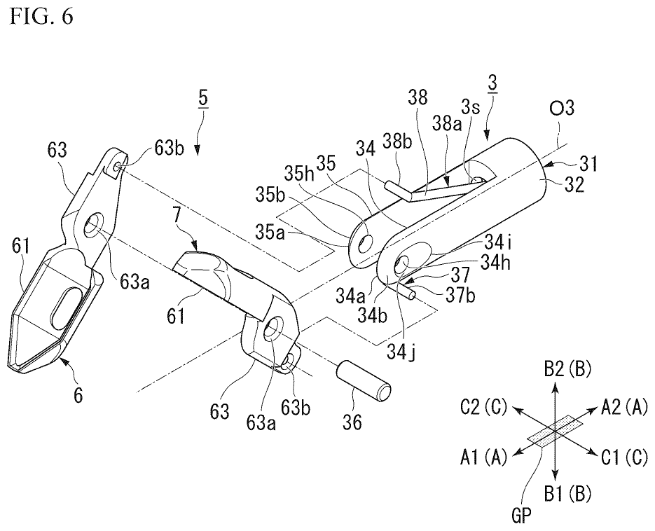

FIG. 4 is a perspective view showing the configuration of the support member 3 and the forceps 5 in a closed state. FIG. 5 is a perspective view showing the configuration of the support member 3 and the forceps 5 in an open state. FIG. 6 is an exploded perspective view showing the configuration of the support member 3 and the forceps 5.

The support member 3 includes a frame 31, a forceps opening/closing pin (a rotating shaft) 36, and a pair of connecting rods 37 and 38. As shown in FIGS. 4 and 5, the forceps 5 has a pair of forceps piece (first forceps piece) 6 and forceps piece (second forceps piece) 7, and the pair of forceps pieces 6 and 7 are supported by the forceps opening/closing pin 36 installed at the frame 31 to be openable and closeable around an axis of the forceps opening/closing pin 36.

### (Frame)

The frame 31 of the support member 3 is formed of a metal such as stainless steel to have an approximately U-shape, as shown in FIG. 6. The frame 31 has a support main body 32 formed in a cylindrical shape and a pair of frame pieces 34 and 35.

The support main body 32 is fixed to the distal end 1a of the sheath 1 shown in FIG. 3 by caulking or the like. As shown in FIGS. 4 and 5, a central axis O3 of the support member 3 in the longitudinal direction A substantially coincides with a central axis O1 of the sheath 1 in the longitudinal direction A.

An internal space 3s of the support main body 32 communicates with the internal space Is of the sheath 1 (FIG. 3).

The operation wire 2 is inserted through the internal space 3s of the support main body 32.

As shown in FIG. 6, the first frame piece 34 and the second frame piece 35 are provided to protrude from the support main body 32 to the distal end side A1. The first frame piece 34 and the second frame piece 35 are provided evenly on both sides in the width direction C with the central axis O3 of the support member 3 interposed therebetween. The first frame piece 34 and the second frame piece 35 are symmetrical with respect to a vertical plane (a plane parallel to the A direction and the B direction) passing through the central axis O3 of the support member 3.

The first frame piece 34 is formed in a flat plate shape that extends in the longitudinal direction A. A plate thickness of the first frame piece 34 in the width direction is similar to a plate thickness of the second frame piece 35. The proximal end side A2 of the first frame piece 34 is continuous with the support main body 32. A semicircular first distal end portion 34a is formed on the distal end side A1 of the first frame piece 34. An inclined surface 34b is formed in an arc shape on the outer surface side of the first distal end portion 34a in the width direction C.

A first through hole 34h penetrating in a plate thickness direction is formed in the distal end side A1 of the first frame piece 34. The first through hole 34h is formed in a bottom surface 34j of a cutout portion 34i that is cut out toward the distal end side A1.

The second frame piece 35 is formed in a flat plate shape that extends in the longitudinal direction A. The proximal end side A2 of the second frame piece 35 is continuous with the support main body 32. A semicircular second distal end portion 35a is formed on the distal end side A1 of the second frame piece 35. An inclined surface 35b is formed on the outer surface side of the second distal end portion 35a in the width direction C.

A second through hole 35h penetrating in the plate thickness direction is formed in the distal end side A1 of the second frame piece 35. The second through hole 35h is formed in a bottom surface (not shown) of a cutout portion (not shown) cut out toward the distal end side A1.

Centers of the first through hole 34h and the second through hole 35h coincide with each other.

### (Forceps Opening/Closing Pin)

The forceps opening/closing pin 36 is made of a metal such as stainless steel and is formed in an approximately cylindrical shape. As shown in FIG. 6, the forceps opening/closing pin 36 is installed in the frame 31 by engaging with the first through hole 34h of the first frame piece 34 and the second through hole 35h of the second frame piece 35.

### (A Pair of Connecting Rods)

The pair of connecting rods 37 and 38 are made of a metal such as stainless steel, as shown in FIG. 6. Each of the connecting rods 37 and 38 has a length in one direction, and the distal end side thereof is bent. The proximal end side of each of the connecting rods 37 and 38 is inserted through the internal space 3s of the support main body 32 of the frame 31 and connected to the operation wire 2. The distal end sides of the connecting rods 37 and 38 are respectively connected to the proximal end sides of the forceps pieces 6 and 7 between the first frame piece 34 and the second frame piece 35.

The connecting rod 37 is engaged with the forceps piece 7 to open and close the forceps piece 7. The connecting rod 37 is disposed on the second frame piece 35 side with respect to the connecting rod 38 within the frame 31. The connecting rod 37 has a first extension portion (not shown) that extends from the proximal end side A2 toward the distal end side A1, and a second extension portion 37b that extends from the distal end side of the first extension portion (not shown) toward the first frame piece 34 side. The distal end side of the second extension portion 37b is engaged with an engagement hole 63b formed on the proximal end side of the forceps piece 7 which will be described below.

The connecting rod 38 is engaged with the forceps piece 6 to open and close the forceps piece 6. The connecting rod 38 is disposed on the first frame piece 34 side with respect to the connecting rod 37 within the frame 31. The connecting rod 38 has a first extension portion 38a that extends from the proximal end side A2 toward the distal end side A1, and a second extension portion 38b that extends from the distal end side of the first extension portion 38a toward the second frame piece 35 side. The distal end side of the second extension portion 38b is engaged with an engagement hole 63b formed on the proximal end side of the forceps piece 6 which will be described below.

### [Forceps 5]

The forceps 5 is a member used to collect biological tissue. As shown in FIGS. 4 and 5, the forceps 5 is made of a metal material such as stainless steel, or the like. The forceps 5 includes a pair of forceps pieces 6 and 7 that face each other when in a closed state.

The forceps piece 6 is supported by the forceps opening/closing pin 36 shown in FIG. 6 to be rotatable around the forceps opening/closing pin 36. The forceps piece 7 is supported by the forceps opening/closing pin 36 shown in FIG. 6 to be rotatable around the forceps opening/closing pin 36. The forceps opening/closing pin 36 is inserted into a first through hole 63a of a first plate 63 of each of the forceps pieces 6 and 7.

The forceps pieces 6 and 7 are operated by the operation wire 2 to which the proximal ends thereof are connected via the connecting rods 37 and 38. The forceps pieces 6 and 7 open and close toward the distal end side A1 in cooperation with each other.

The forceps pieces 6 and 7 are disposed symmetrically with respect to a central axis O5 of the forceps 5 in the longitudinal direction A. The central axis O5 of the forceps 5 in the longitudinal direction A substantially coincides with the central axis O1 of the sheath 1 in the longitudinal direction A. The central axis O5 of the forceps 5 in the longitudinal direction A coincides with grip surfaces GP that face each other when the forceps pieces 6 and 7 are in a closed state.

Each of the forceps pieces 6 and 7 has a tissue accommodating recess (a recess) 65 capable of collecting biological tissue. When the first forceps piece 6 and the second forceps piece 7 are in the closed state, the tissue accommodating recesses 65 face each other. The tissue accommodating recess 65 of the forceps piece 6 and the tissue accommodating recess 65 of the forceps piece 7 face each other substantially entirely on the opening sides thereof when the forceps 5 are in the closed state. A size of the tissue accommodating recess 65 formed in each of the forceps pieces 6 and 7 is preferably set so that a volume of a space formed by the tissue accommodating recess 65 when the forceps 5 is in the closed state, that is, an amount of tissue collected by the forceps 5, is within a range of 0.7 mm³ or more and 6.0 mm³ or less.

Further, a maximum outer diameter of the forceps 5 in a direction (the width direction C and the up-down direction B) intersecting the central axis O5 is smaller than an outer diameter of the sheath 1. The maximum outer diameter of the forceps 5 around the central axis O5 is 2.2 mm or less, and more preferably in a range of 1.2 mm or more and 1.8 mm or less, and is about 1.8 mm in this embodiment.

The forceps 5 are installed at the distal end of the sheath 1 which is inserted into the channel 216 shown in FIG. 1, and can be advanced and retracted in the longitudinal direction of the channel 216. It is preferable that the forceps 5 be sized so that the distal end side A1 of the forceps 5 does not come into contact with an inner surface of the channel 261 when the forceps 5 is advanced within the channel 216.

The forceps pieces 6 and 7 have the same rotation range in the up-down direction B with respect to the central axis O5, but the rotation range of the forceps pieces 6 and 7 with respect to the central axis O5 may be different. Moreover, the forceps piece 7 may be fixed to the support member 3, and only the forceps piece 6 may be rotatably supported by the support member 3. The forceps piece 7 may be a cover member that does not have the tissue accommodating recess 65.

### (Forceps Piece 6, Forceps Piece 7)

The forceps piece 6 and the forceps piece 7 that constitute the forceps 5 of this embodiment have the same configuration, and thus in the following description, only the forceps piece 6 will be described.

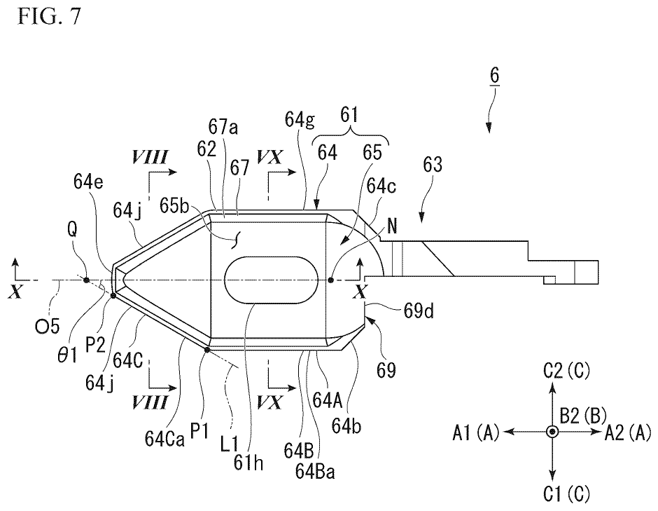

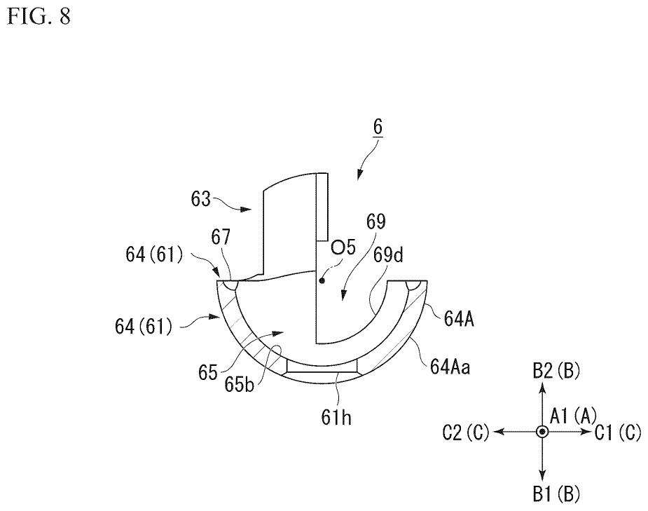

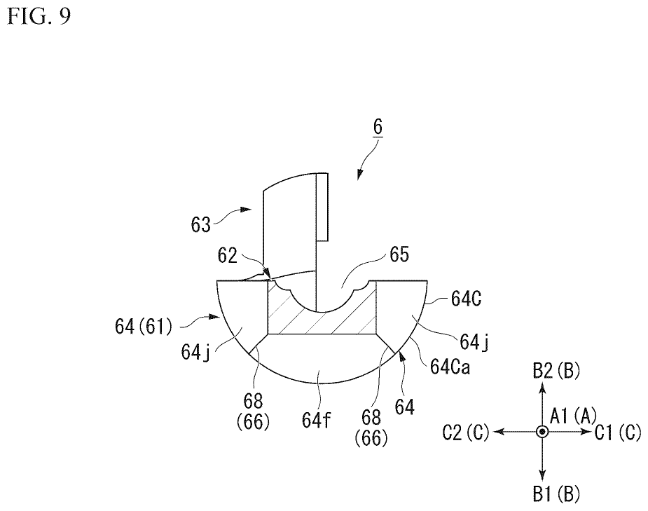

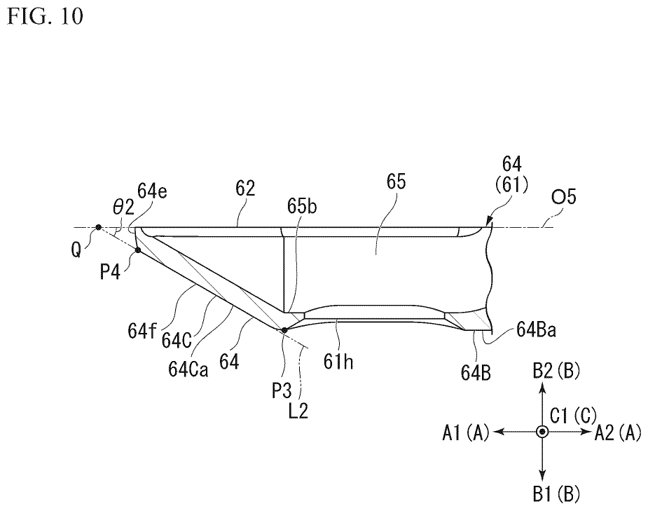

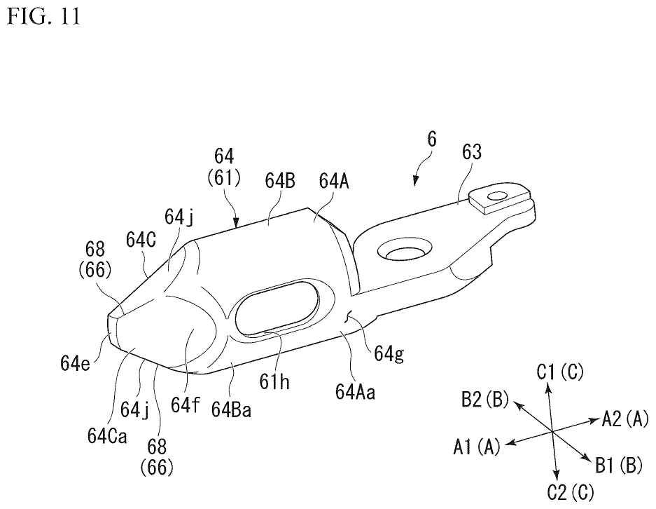

FIG. 7 is a plan view showing the configuration of the forceps piece (the first member) 6. FIG. 8 is a cross-sectional view taken along line VIII-VIII in FIG. 7. FIG. 9 is a cross-sectional view taken along line VX-VX in FIG. 7. FIG. 10 is a cross-sectional view taken along line XX in FIG. 7. FIG. 11 is a perspective view showing the configuration of the forceps pieces 6.

As shown in FIG. 7, the forceps piece 6 is formed from a single flat plate mainly by cutting or pressing. The forceps piece 6 has a forceps cup 61 provided on the distal end side A1 in the longitudinal direction A and a first plate 63 provided on the proximal end side A2 in the longitudinal direction A.

As shown in FIG. 8, a cross section of the forceps cup 61 perpendicular to the longitudinal direction A is formed in an approximately hemispherical shape, and is open to the forceps piece 7 side (the upper side B2) shown in FIGS. 4 and 5 in the opening-closing direction B. The forceps cup 61 has a cup main body 64 and the tissue accommodating recess 65 formed inside the cup main body 64.

The cup main body 64 has three portions (a proximal end portion 64A, a central portion (a body portion) 64B, and a distal end portion 64C) arranged from the first plate 63 side in order in the longitudinal direction A.

The proximal end portion 64A is a portion located closest to the proximal end side A2 of the cup main body 64 and connected to the first plate 63. A cutout portion 69 that penetrates from a vicinity of the center in the width direction to one side in the width direction (the right side C1) is formed on the proximal end side A2 of the proximal end portion 64A. The cutout portion 69 partially shortens a length of the right side C1 of the proximal end portion 64A in an axial direction. The cutout portion 69 reaches the tissue accommodating recess 65 and cuts out the proximal end side A2 of the tissue accommodating recess 65. Therefore, when seen in the axial direction shown in FIG. 8, an opening 69d having a sector shape is formed on the proximal end side A2 of the proximal end portion 64A. Through the opening 69d, the tissue accommodating recess 65 communicates with the outside.

Further, in the proximal end portion 64A, a pair of cutout surfaces 64b and 64c is formed on both sides of an outer circumferential surface in the width direction C. The cutout surfaces 64b and 64c are inclined surfaces that approach the central axis O5 from a vicinity of the center of the proximal end portion 64A in the longitudinal direction A toward the proximal end side A2 when seen from the opening side of the tissue accommodating recess 65 shown in FIG. 7. Due to the presence of the cutout portion 69, the first cutout portion surface 64b located on one side in the width direction (right C1 side) is located on the distal end side A1 with respect to the second cutout portion surface 64c.

As shown in FIG. 7, when seen from the opening side of the forceps pieces 6, an opening-side outer circumferential edge of the proximal end portion 64A, excluding the cutout portion 69, forms an arc shape centered on a point N that overlaps the central axis O5. In this embodiment, the cutout surfaces 64b and 64c are provided on the proximal end side A2 of the proximal end portion 64A to an extent that a part of the opening-side outer circumferential edge can maintain an arc shape, thereby preventing the proximal end side A2 of the cup main body 64 from having a sharp shape. In the proximal end portion 64A, a shape of an inner circumferential surface which is formed as a part of a sphere is different from a shape of an outer circumferential surface.

As shown in FIGS. 7 and 8, the central portion (the body portion) 64B has a semi-cylindrical shape and has a constant width in the longitudinal direction A. When seen from the opening side of the forceps pieces 6 shown in FIG. 7, opening-side outer circumferential edges of the central portion 64B on both sides in the width direction are approximately parallel to the central axis O5. In the central portion 64B, a shape of an inner circumferential surface and a shape of an outer circumferential surface are substantially the same, and are curved around the central axis O5.

As shown in FIGS. 7 and 11, the distal end portion 64C is tapered from the central portion 64B side toward the distal end side A1. In this embodiment, a width of the distal end portion 64C in the direction (the width direction C, the up-down direction B) perpendicular to the longitudinal direction A gradually decreases toward the distal end side A1.

An outer circumferential surface 64Ca of the distal end portion 64C is configured of a plurality of inclined surfaces. In this embodiment, the outer circumferential surface 64Ca of the distal end portion 64C has a pair of inclined surfaces 64j formed on both sides in the width direction C, and an inclined surface 64f (FIG. 11) located between the pair of inclined surfaces 64j and on the lower side (the B1 side) intersecting the central axis O5. In this embodiment, the three inclined surfaces 64j and 64f are formed on the outer circumferential surface of each of the forceps pieces 6 and 7, but the number of inclined surfaces is not limited to three. However, it is preferred that at least two intersecting ridge portions 68 be formed. In addition, in this embodiment, the inclined surfaces 64j and 64f of each of the forceps pieces 6 and 7 are all flat surfaces, but at least one of the plurality of inclined surfaces may be a curved surface.

As shown in FIG. 7, the pair of inclined surfaces 64j are inclined in directions (the C1 side, the C2 side) approaching the central axis O5 as they approach the distal end side A1, and thus a width dimension of the distal end portion 64C in the width direction C is reduced. As shown in FIG. 10, the inclined surface 64f is inclined in a direction (the B2 side) approaching the central axis O5 toward the distal end side A1, and thus a width dimension of the distal end portion 64C in the up-down direction B becomes smaller.

In this embodiment, for example, as shown in FIGS. 7 and 10, angles θ1 and θ2 of the inclined surfaces 64j and 64f with respect to the central axis O5 are both approximately 30°. The both angles θ1 and θ2 are 30°, but are not limited thereto and are preferably within a range of, for example, 15° to 45°.

In addition, a shape of the distal end portion 64C in this embodiment is symmetrical on both sides in the width direction with respect to the central axis O5, but it may be asymmetrical. Furthermore, the angles θ1 and θ2 of the inclined surfaces 64j and 64f with respect to the central axis O5 may be different from each other.

On the outer circumferential surface of the distal end portion 64C, linear intersecting ridge portions 68 are formed at the boundaries between the pair of inclined surfaces 64j and 64f described above. When seen from the distal end side A1, the intersecting ridge portion 68 between the inclined surface 64j and the inclined surface 64f and the intersecting ridge portion 68 on one side and the intersecting ridge portion 68 between the inclined surface 64j and the inclined surface 64f on the other side extend in directions away from each other from the distal end side A1 toward the proximal end side A2. Additionally, a boundary between the three inclined surfaces 64j and 64f of the distal end portion 64C and the outer circumferential surface 64Ba of the central portion 64B has a smoothly curved shape.

A distal end surface 64e that intersects the central axis O5 and faces the distal end side A1 is formed on the most distal end side A1 of the cup main body 64. The distal end surface 64e has a rectangular shape with a length in the width direction C when seen from the distal end side in the axial direction. The distal end surface 64e is slightly curved toward the distal end side A1.

As shown in FIG. 7, when a first straight line that connects a first point P1 located at a boundary between the outer circumferential surface 64Ba of the central portion 64B and the outer circumferential surface 64Ca (the inclined surface 64j) of the distal end portion 64C and a second point P2 located at a boundary between the outer circumferential surface 64Ca (the inclined surface 64j) of the distal end portion 64C and the distal end surface 64e is defined as L1, the distal end surface 64e is located on the proximal end side A2 with respect to a position of an intersection point Q at which the first straight line L1 and the central axis (the axis) O5 intersect.

Further, as shown in FIG. 10, when a second straight line that connects a third point (the first point) P3 located at a boundary between the outer circumferential surface 64Ba of the central portion 64B and the outer circumferential surface 64Ca (the inclined surface 64f) of the distal end portion 64C, and a fourth point (the second point) P4 located at a boundary between the outer circumferential surface 64Ca (the inclined surface 64f) of the distal end portion 64C and the distal end surface 64e is defined as L2, the distal end surface 64e is located on the proximal end side A2 with respect to a position of an intersection point Q at which the second straight line L2 and the central axis O5 intersect.

As shown in FIGS. 7 and 10, although the points at which each of the first line L1 and the second line L2 intersects the central axis O5 are both the intersection points Q, the points at which each of the first line L1 and the second line L2 intersects the central axis O5 may be different from each other. In other words, the angles θ1 and θ2 formed between the central axis O5 and each of the inclined surfaces 64j and 64f may be different.

In this embodiment, the first straight line L1 is a straight line that extends along the inclined surface 64j, and the second straight line L2 is a straight line that extends along the inclined surface 64f. Therefore, the angles of the first straight line L1 and the second straight line L2 with respect to the central axis O5 are equal to the inclination angles (θ1 and θ2) of the inclined surfaces 64j and 64f described above, and are all 30°.

The tissue accommodating recess 65 is recessed downward (toward the B1 side) from the grip surface GP in a direction away from the forceps pieces 7 in an opening and closing direction of the forceps pieces 6 with respect to the forceps pieces 7. The tissue accommodating recess 65 is formed over substantially the entirety of the cup main body 64 in the longitudinal direction A. That is, the tissue accommodating recess 65 is formed inside three portions (the proximal end portion 64A, the central portion 64B, and the distal end portion 64C) that constitute the cup main body 64.

As shown in FIG. 7, the cup main body 64 having such a shape has an outer circumferential blade 62 formed on an outer circumferential edge on the opening side, a groove portion 67 formed adjacent to the outer circumferential blade 62 and inward with respect to the outer circumferential blade 62, and the through hole 61h that penetrates in the opening and closing direction B in the vicinity of a center of a bottom portion 65b of the tissue accommodating recess 65.

The outer circumferential blade 62 is formed continuously on an outer circumferential edge of the cup main body 64 over at least the distal end portion 64C and the central portion 64B. A range in which the outer circumferential blade 62 is formed is not limited to the range described above, and may be formed, for example, continuously from the distal end portion 64C and the central portion 64B to the base portion 64A. It is preferable that the outer circumferential blade 62 be formed with a thickness that allows it to cut biological tissue well and prevents the blade from chipping.

The groove portion 67 is formed on the outer circumferential edge side of the opening side of the cup main body 64. As shown in FIGS. 7 and 8, the groove portion 67 is formed in a concave shape that is recessed toward the side (the B2 side) opposite to the forceps pieces 7 with respect to the outer circumferential blade 62, and a cross section that intersects an extension direction thereof has an arc shape. The groove portion 67 is formed at least in a range in which the circumferential cutting edge 62 is formed. By forming the groove portion 67 on the outer circumferential edge of the opening side of the cup main body 64, sharpness of the outer circumferential blade 62 is ensured while a thickness of a wall portion of the cup main body 64 is ensured, thereby ensuring rigidity of the cup main body 64.

The through hole 61h is formed in the central portion 64B located at the center of the cup main body 64 in the longitudinal direction A. The through hole 61h is an elongated hole in the longitudinal direction A at the center of the bottom portion of the cup main body 64, and the distal end side A1 and the proximal end side A2 thereof are semicircular. Through the through hole 61h, the tissue accommodating recess 65 communicates with the outside. A size (an opening area), a shape, and the like of the through hole 61h are set according to flexibility of the biological tissue.

As shown in FIGS. 9 and 11, the cup main body 64 of this embodiment has an anti-slip portion 66 on the outer circumferential surface side for preventing the forceps 5 (the forceps pieces 6 and 7) from slipping against the biological tissue. In this embodiment, there are two anti-slip portions 66 on the outer circumferential surface of the distal end portion 64C of the cup main body 64. Each of the anti-slip portions 66 is configured of the intersecting ridge portion 68 formed on the outer circumferential surface of the distal end portion 64C of the cup main body 64. The anti-slip portion 66 (the intersecting ridge portion 68) is a protruding portion that protrudes most radially outward on the outer circumferential surface of the distal end portion 64C at a predetermined position in the longitudinal direction. Therefore, the anti-slip portions 66 (the intersecting ridge portion 68) become portions with the largest coefficient of friction on the outer circumferential surfaces of the forceps pieces 6 and 7, and when the forceps 5 is pressed against biological tissue, the anti-slip portions 66 come into contact with the biological tissue (mucosal tissue), and appropriate hooking occurs, thereby making the forceps 5 less likely to slip. Thus, it is possible to stabilize a position of the forceps 5 with respect to the biological tissue. Thus, in this embodiment, a plurality of intersecting ridge portions 68 formed on the outer circumferential surfaces of the forceps pieces 6 and 7 function as the anti-slip portions 66 of the forceps 5 with respect to the biological tissue.

In this embodiment, a pair of intersecting ridge portions 68 formed at each of the boundaries between the plurality of inclined surfaces 64j and 64f formed on the outer circumferential surface of the distal end portion 64C are used as the anti-slip portion 66, but the number and shapes of the inclined surfaces and intersecting ridge portions are not limited to the configuration shown in the drawing.

### [Method for Collecting Tissue Using Endoscopic Treatment System 300]

Next, a procedure using the endoscopic treatment system 300 of this embodiment shown in FIGS. 1 and 2, that is, a method for collecting tissue using the endoscopic treatment system 300 will be described. Specifically, an operation of the biopsy forceps 100 used in diagnosing lung cancer and for collecting target tissue (lymph nodes) will be described.

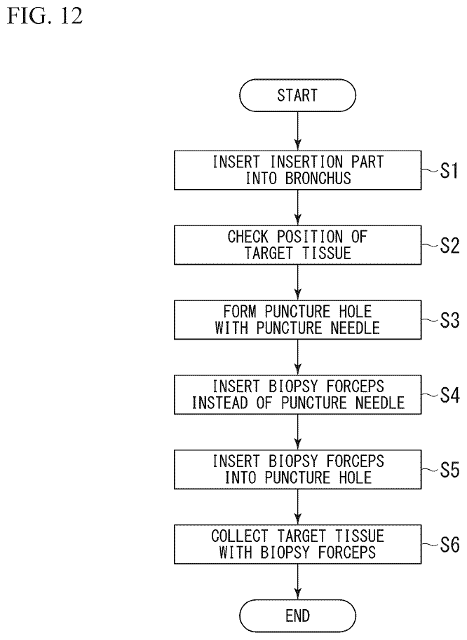

FIG. 12 is a flow chart showing a method for collecting tissue using the endoscopic treatment system 300. A method of using the endoscopic treatment system 300 will be described with reference to this FIG. 12.

First, the user confirms a position of biological tissue in which a lesion or the like has occurred using an X-ray fluoroscopic image or the like, and inserts the insertion part 210 into a trachea while checking with the imaging part 213 of the ultrasonic endoscope 200 (Step S1).

The user bends or curves the distal end portion 211 of the insertion part 210 while rotating the input part 222 of the operation part 220, selects a bronchus to be penetrated while checking the branching with the ultrasonic endoscope 200, and advances the insertion part 210. At this time, the target tissue (the lymph node) to be collected as a tissue sample is located outside the bronchus and cannot be seen from inside the bronchus. Therefore, a position of the target tissue (the lymph node) is captured by the ultrasonic endoscope 200. When the ultrasonic endoscope 200 is brought close to the target tissue (the lymph node), a part appears to change color. This part is the target tissue (the lymph node). In this way, the position of the target tissue is confirmed from inside the bronchus (Step S2).

When the user has confirmed the position of the target tissue (the lymph node) using the ultrasonic endoscope 200, he or she stops the advancement of the insertion part 210 and protrudes the puncture needle from the channel 216 of the distal end portion 211 to puncture a bronchial wall. The user continues the puncturing until the puncture needle penetrates the bronchial wall and reaches the target tissue (the lymph node). At this time, the user uses the ultrasonic endoscope 200 to confirm that the puncture needle has reached the target tissue (the lymph node). In this manner, puncture holes are formed in the bronchial wall and the target tissue (the lymph node) (Step S3). At this time, the position at which the puncture hole is formed may be marked on the bronchial wall.

Then, the user removes the puncture needle from the channel 216, inserts the biopsy forceps 100 into the channel 216, and causes the biopsy forceps 100 to protrude from the distal end portion 211 of the insertion part 210 (Step S4).

The user operates the operation part 8 to bring the biopsy forceps 100 close to the vicinity of the puncture hole (the marking) formed in the bronchial wall, and inserts the distal end of the biopsy forceps 100 into the puncture hole while keeping the biopsy forceps 100 in the closed state. Then, the biopsy forceps 100 is further advanced toward the target tissue (the lymph node) and inserted into the puncture hole formed in the target tissue (the lymph node) (Step S5).

After the user confirms with the ultrasonic endoscope 200 that the biopsy forceps 100 has reached the puncture hole in the target tissue (the lymph node), the user operates the operation part 8 to open and close the biopsy forceps 100, thereby collecting a portion of the target tissue (the lymph node) (Step S6).

Typically, a size of the puncture hole is about 0.5 mm to 1.1 mm.

In contrast, a maximum diameter of the forceps 5 (the central portion 64B) of this embodiment is about 1.8 mm. By increasing the outer diameter of the forceps 5 of the biopsy forceps 100, it is possible to form a large tissue storage recess 65 on the inside of each of the forceps pieces 6 and 7, and thus it is possible to ensure a sufficient amount of tissue to be collected for the examination.

On the other hand, when considering the insertability of the forceps pieces 6 and 7 into the puncture hole, if the maximum diameter parts of the forceps pieces 6 and 7 are at the distal end sides A1 of the forceps pieces 6 and 7, it becomes difficult to insert the forceps 5 into the puncture hole. Therefore, in the forceps 5 of this embodiment, the distal end side A1 of each of the forceps pieces 6 and 7 is formed to be thin and non-sharp, and a minimum width thereof is smaller than the diameter of the puncture hole. In this embodiment, since the distal end side A1 of each of the forceps pieces 6 and 7 is rounded rather than sharp as in the conventional case, the distal end side of the forceps 5 does not pierce the target tissue, and the target tissue can be collected in good quality without blood being mixed into the collected tissue.

In addition, in each of the forceps pieces 6 and 7 of this embodiment, the through hole 61h is formed in the central portion 64B of the cup main body 64 which is a widest portion, and thus a part of the target tissue gripped by the forceps 5 and accommodated in the tissue accommodating recess 65 can be allowed to escape slightly to the outside through the through hole 61h. A space is created in the tissue accommodating recess 65 by an amount of tissue pushed out of the through hole 61h, and thus it is possible to grip and collect a larger amount of target tissue.

Therefore, when the forceps 5 of the biopsy forceps 100 is inserted into the puncture hole from the distal end side A1, the diameter of each of the puncture holes formed in the bronchial wall and the target tissue (the lymph node) can be gradually widened by the forceps 5 of the biopsy forceps 100, while the entire biopsy forceps 100 can be inserted into each of the puncture holes. Since the bronchial wall and the target tissue (the lymph node) are flexible, even if the forceps 5 is slightly larger than the puncture hole, it is possible to widen the puncture hole and insert the forceps 5 into the hole. In this manner, the forceps 5 of this embodiment can ensure a sufficient collection amount of tissue as a tissue sample, and also has excellent insertability into the puncture hole.

Furthermore, conventional forceps tend to slip on the bronchial wall and the tissue surface of the target tissue (the lymph node). In particular, since the target tissue (the lymph node) is more flexible than the bronchial wall, the forceps tend to slip on the tissue surface near the puncture hole, and thus it may be difficult to insert the forceps into the puncture hole.

In contrast, the forceps 5 of this embodiment has the plurality of inclined surfaces 64j and 64f formed on the outer circumferential surface of the tapered distal end side A1, and has the plurality of anti-slip portions 66 formed of the intersecting ridge portions 68 thereof. Since the plurality of anti-slip portions 66 that protrude radially outward with respect to the inclined surfaces 64j and 64f are present at substantially equal intervals around the central axis O5 of the forceps 5, it is possible to curb rotation of the forceps 5 around the axis thereof and stabilize a posture of the forceps 5 by hooking onto the bronchial wall or the tissue surface of the target tissue (the lymph node). Thus, since the forceps 5 can be smoothly inserted into the puncture hole, operability can be improved.

In the forceps 5 of this embodiment, the outer circumferential blades 62 are formed along the outer circumferential edge portion of the opening side of each of the forceps pieces 6 and 7, and when the forceps 5 is in the closed state, the outer circumferential blades 62 of the forceps pieces 6 and 7 are engaged with each other at a position parallel to the grip surface GP. Therefore, by closing the forceps 5, a part of the target tissue (the lymph node) can be collected with a clean cut surface. In this way, it is possible to prevent tissue contamination and surface damage during collection, to reduce crushing of a sample and blood contamination, and to enable the target tissue to be collected in a high-quality sample, thereby enabling an accurate test using the collected tissue.

In this embodiment, the forceps 5 are advanced to the inside of the target tissue using the puncture hole and are then opened and closed inside the target tissue to collect the tissue. Therefore, it is possible to collect a sufficient amount of only the target tissue in one operation. Conventionally, when the puncture needle is a suction biopsy needle, it is possible to determine whether a lesion is present in the target tissue by collecting a tissue sample when the puncture needle is inserted into the target tissue, but when the amount of tissue sample collected by the puncture needle is small and insufficient for performing a genetic test, or the like, it has been necessary to repeatedly perform the puncturing to collect more tissue. In contrast, since the biopsy forceps of the present disclosure does not require multiple approaches to the target tissue, there is no risk of contamination with other tissues, making it possible to ensure sufficient quality tissue as a sample and reducing a burden of a collection operation.

### Second Embodiment

Next, a configuration of forceps pieces (a first forceps piece, a second forceps piece) according to a second embodiment of the present disclosure will be described. In the following description, the description of the same configuration as in the above embodiment will be omitted, and only the different parts will be described in detail.

FIG. 12 is a perspective view showing a forceps piece 76 of the second embodiment.

The forceps piece of the second embodiment differs from that of the above embodiment in the shape of the distal end side A1. A forceps is formed by combining a forceps piece 76 shown in FIG. 12 with another forceps piece having a similar shape in the up-down direction. Since a pair of forceps pieces constituting the forceps have the same configuration, only one forceps piece 76 will be described in detail here.

The forceps piece 76 has a plurality of anti-slip portions 66 and 79 on an outer circumferential surface of a distal end portion 74C of a cup main body 74. As described above, a first anti-slip portion (the anti-slip portion) 66 is configured of the pair of intersecting ridge portions 68 formed on the outer circumferential surface of the distal end portion 74C. In this embodiment, the anti-slip portion 66 is referred to as the first anti-slip portion 66. A second anti-slip portion (the anti-slip portion) 79 is configured of three recesses (a pair of recesses 73j and 73f) formed in the outer circumferential surface of the distal end portion 74C. The first anti-slip portion 66 and the second anti-slip portion 79 make the outer circumferential surface of the distal end portion 74C surface that has greater shape change due to unevenness than in the first embodiment.

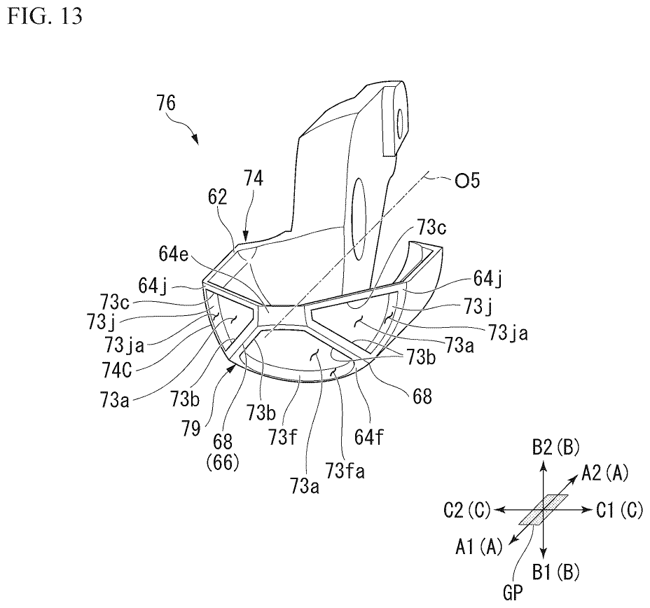

As shown in FIG. 13, on the outer circumferential surface of the distal end portion 74C, the intersecting ridge portions 68 are formed at boundaries between the three inclined surfaces 64j and 64f in the same manner as in the above embodiment to protrude radially outward. The recesses 73j and 73f and the intersecting ridge portions 68 are alternately arranged around the central axis O5.

On the outer circumferential surface of the distal end portion 74C, each of the inclined surfaces 64j on both sides in the width direction is formed with the recess 73j, and the remaining inclined surface 64f is formed with the recess 73f. The inclined surfaces 64j and 64f form a frame shape around the recesses 73j and 73f. That is, in this embodiment, a columnar ridge portion is formed along an outer circumferential edge of each of the inclined surfaces 64j and 64f.

These three recesses 73j and 73f are recesses recessed inward from the respective inclined surfaces 64j and 64f, and bottom surfaces 73a thereof are approximately parallel to the inclined surfaces 64j and 64f. Depths of the three recesses 73j and 73f may be the same or different as long as the wall thickness is sufficient to ensure the rigidity of the cup main body 74.

An opening edge of the recess 73j is formed along an intersecting ridge between the frame-shaped inclined surface 64j and a recess inner wall surface 73ja that is substantially perpendicular to the inclined surface 64j. An opening edge of the recess 73f is formed along an intersecting ridge between the frame-shaped inclined surface 64f and a recess inner wall surface 73fa that is substantially perpendicular to the inclined surface 64f.

The opening edges of the recesses 73j and 73f are partially formed linearly along the intersecting ridge portion 68 or the grip surface GP. Opening edge portions 73b, which are part of the opening edges of the recesses 73j and 73f, are substantially parallel to the intersecting ridge portion 68. Each of the opening edge portion 73c, which is a part of the opening edge of the recess 73j, is approximately parallel to the grip surface GP. The linear opening edge portions 73b and 73c are spaced apart from each other around the central axis O5 and extend radially outward from the central axis O5 when seen from the distal end side A1.

According to the forceps piece 76 of this embodiment, in addition to the intersecting ridge portion 68 formed on the outer circumferential surface of the distal end side A1, the plurality of recesses 73j and 73f are provided on each of the inclined surfaces 64j and 64f, thereby increasing the unevenness on the outer circumferential surface and improving an anti-slip effect on biological tissue compared to the configuration of the first embodiment. When the forceps equipped with such forceps pieces 76 are brought into contact with biological tissue, a part of the biological tissue enters each of the recesses 73j and 73f of each of the forceps pieces 76, and when the forceps piece 76 is about to start slipping, the biological tissue gets caught on the opening edge portion 73b of each of the recesses 73j and 73f and the intersecting ridge portion 68. In this way, the second anti-slip portion 79 and the first anti-slip portion 66 of the forceps piece 76 come into contact with the biological tissue, and appropriate hooking occurs, thereby making the forceps piece 76 less likely to slip and making it possible to stabilize the position of the forceps. In this embodiment, the inclined surfaces 64j and 64f surrounding the recesses 73j and 73f are frame-shaped to define the recesses 73j and 73f. Therefore, the forceps pieces 76 are prevented from slipping in the longitudinal direction A and the width direction C by being caught on the biological tissue that has entered the recesses 73j and 73f.

### Third Embodiment

Next, a configuration of forceps pieces (a first forceps piece, a second forceps piece) of a third embodiment of the present disclosure will be described. In the following description, the description of the same configuration as in the above embodiment will be omitted, and only the different parts will be described in detail.

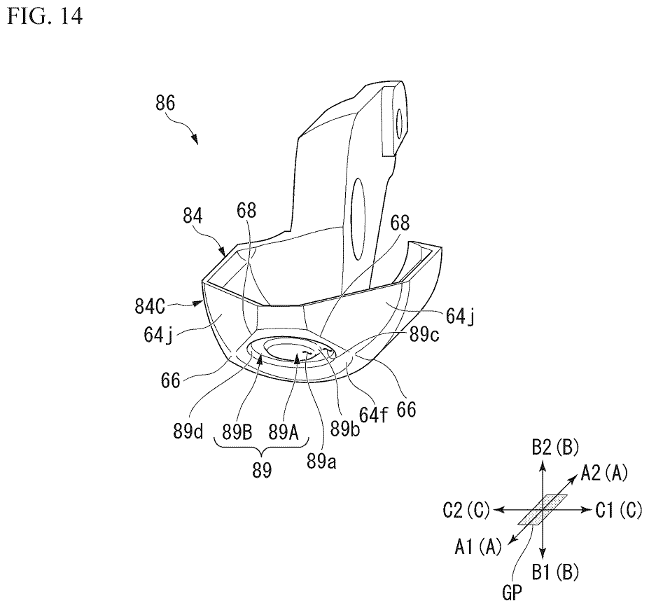

FIG. 14 is a perspective view showing a forceps piece 86 of the third embodiment.

The shape of the distal end side A1 of the forceps piece 86 of the third embodiment differs from that of the above embodiments. A forceps is formed by combining the forceps piece 86 shown in FIG. 14 with another forceps piece having a similar shape in the up-down direction. Since a pair of forceps pieces constituting the forceps have the same configuration, only one forceps piece 86 will be described in detail here.

The forceps piece 86 has a plurality of anti-slip portions 66 and 89 on an outer circumferential surface of a distal end portion 84C of a cup main body 84. As described above, the anti-slip portion 66 is configured of a pair of intersecting ridge portions 68 formed on the outer circumferential surface of the distal end portion 84C. In this embodiment, the anti-slip portion 66 is also referred to as a first anti-slip portion 66. A second anti-slip portion (the anti-slip portion) 89 is formed on the inclined surface 64f that is the furthest from the opening side and has a largest area among the three inclined surfaces 64j and 64f formed on the outer circumferential surface of the distal end portion 84C. The second anti-slip portion 89 has a circular central protrusion 89A and an annular recess 89B surrounding the circumference of the central protrusion 89A.

The central protrusion 89A is located approximately at the center of the inclined surface 64f. A circumferential wall surface 89b of the central protrusion 89A is a surface that connects a circular flat surface 89a and a bottom surface 89c of the annular recess 89B, and is a curved surface that curves radially outward of the circular flat surface 89a. The circumferential wall surface 89b may be, for example, an inclined surface that obliquely connects the circular flat surface 89a and the bottom surface 89c. In this case, the inclined surface is inclined in a direction (in a direction in which a width of the annular recess 89B is narrowed) in which the inclined surface widens radially outward of the circular flat surface 89a from the circular flat surface 89a to the bottom surface 89c. A shape of the circumferential wall surface 89b of the central protrusion 89A may be a curved surface or an inclined surface as described above, so long as an intersection between the circular plane 89a and the circumferential wall surface 89b does not have a sharp shape.

Alternatively, an outer circumferential edge portion (an outer circumferential edge portion of the circular flat surface 89a) of the central protrusion 89A on the protruding side may be, for example, R-chamfered. In other words, an intersection portion between the circular flat surface 89a, which is approximately parallel to the inclined surface 64f, and the circumferential wall surface 89b may be a curved surface.

The annular recess 89B is a recess formed in an annular shape with a constant width and a constant depth around the central protrusion 89A. An inner circumferential surface 89d of the annular recess 89B and the inclined surface 64f intersect each other substantially perpendicularly or at an obtuse angle, and an intersecting ridge is formed at a boundary therebetween. The intersecting ridge forms a circle when seen from a normal direction of the inclined surface 64f. The annular recess 89B is preferably formed inward in the width direction with respect to the pair of intersecting ridges 68 and has a size that does not reach each of the intersecting ridges 68.

The size, shape, and number of the central protrusions 89A and the annular recesses 89B are not limited to those shown in the drawing and can be modified as appropriate.

According to the forceps piece 86 of this embodiment, in addition to the intersecting ridge portion 68 formed on the outer circumferential surface on the distal end side A1, the central protrusion 89A and the annular recess 89B are provided on the inclined surface 64f, which has the largest area, thereby increasing the unevenness on the outer circumferential surface and improving the anti-slip effect on biological tissue compared to the configuration of the first embodiment.

When the forceps including the forceps piece 86 having the second anti-slip portion 89 is brought into contact with biological tissue, a part of the biological tissue enters the annular recess 89B, and when the forceps pieces 86 is about to start slipping, the inner circumferential surface and the opening-side outer circumferential edge of the annular recess 89B, and the circumferential wall surface 89b and the protruding-side outer circumferential edge of the central protrusion 89A get caught on the biological tissue.

In this way, the second anti-slip portion 79 and the first anti-slip portion 66 of the forceps piece 86 come into contact with the biological tissue, and appropriate hooking occurs, thereby making the forceps piece 86 less likely to slip, and making it possible to stabilize the position of the forceps.

In the second anti-slip portion 89 of this embodiment, since a part of the biological tissue that has entered the annular recess 89B can be partially pressed by the central protrusion 89A, it is possible to prevent the biological tissue that has entered the annular recess 89B from slipping out of the recess.

An uneven surface of the second anti-slip portion 89 of the forceps piece 86 also functions as an uneven portion that diffusely reflects ultrasonic waves. That is, ultrasonic waves emitted from a ultrasonic transducer of the ultrasonic endoscope 200 are diffusely reflected by the uneven surface of the second anti-slip portion (the uneven portion) 89 of the forceps piece 86. Some of the diffusely reflected ultrasonic waves are received by the ultrasonic transducer, and it is possible to clearly display the distal end of the forceps piece 86 on a ultrasonic tomographic image. In this way, the second anti-slip portion 89 also greatly contributes to improving visibility of the forceps pieces 86 under ultrasonic images. Thus, it is easier to check opening and closing movement and a gripping state of the forceps.

### Fourth Embodiment

Next, a configuration of forceps pieces 96, 97 (a first forceps piece, a second forceps piece) of a fourth embodiment of the present disclosure will be described. In the following description, the description of the same configuration as in the above embodiment will be omitted, and only the different parts will be described in detail.

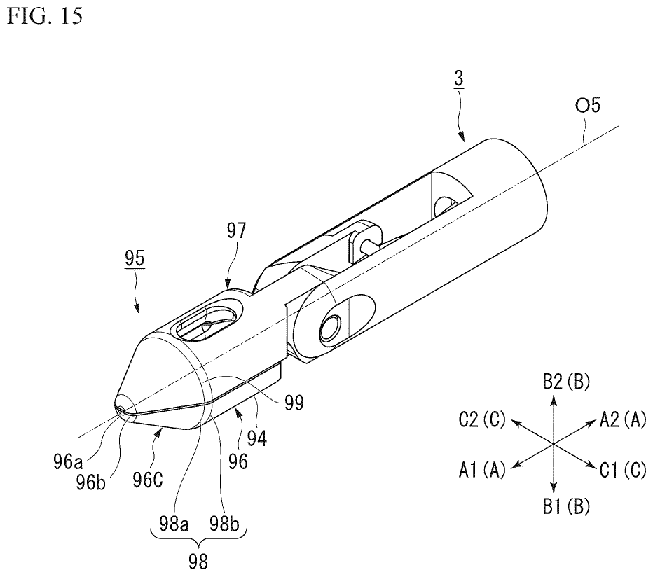

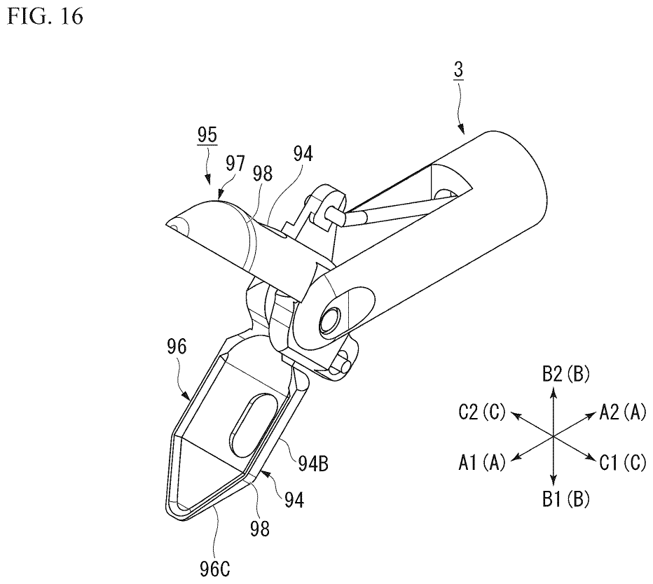

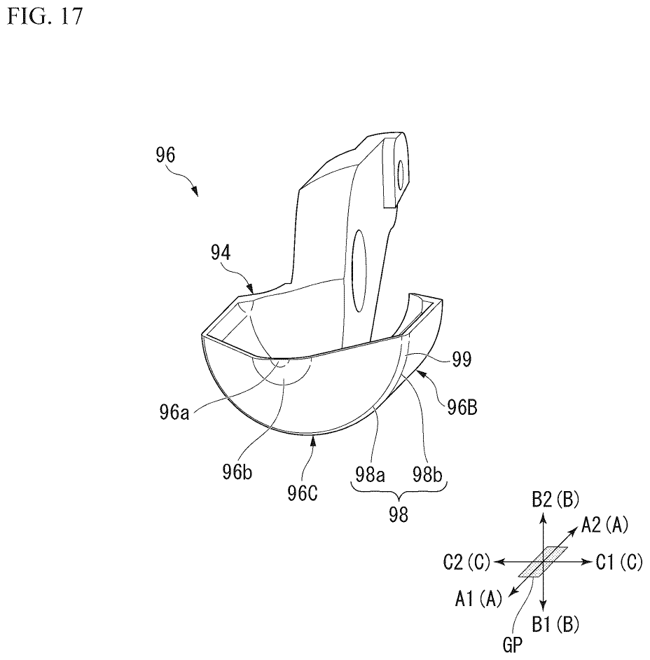

FIG. 15 is a perspective view showing a forceps 95 in a closed state having forceps pieces 96 and 97 of the fourth embodiment. FIG. 16 is a perspective view showing the forceps 95 in an open state having the forceps pieces 96 and 97 of the fourth embodiment. FIG. 17 is a perspective view showing the forceps piece 96 of the fourth embodiment.

In the fourth embodiment, a shape of the distal end side A1 of the forceps pieces 96 and 97 is also different from that of the above-described embodiments. Since the forceps pieces 96 and 97 of this embodiment have the same configuration, only one forceps piece 96 will be described in detail here.

As shown in FIGS. 15 to 17, the forceps piece 96 of this embodiment has a cross-sectional shape intersecting the axial direction that is semicircular at any position in the axial direction. A size of the semicircular shape of the forceps piece 96 gradually decreases toward the distal end side A1. That is, a distal end portion 96C of the forceps piece 96 is shaped so that a width of a cross section perpendicular to the longitudinal direction gradually decreases around the central axis O5 from a central portion 94B toward the distal end side A1. That is, the distal end portion 96C is shaped so that a radial dimension about the central axis O5 gradually decreases from the central portion 94B toward the distal end side A1.

A distal end surface 96a that has a semicircular shape and intersects the axial direction is formed on the most distal end side A1 of the forceps piece 96. A curved surface 96b curved radially outward is formed around the distal end surface 96a.

The forceps piece 96 has an anti-slip portion 98 at a boundary between the distal end portion 96C and a central portion 94B of a cup main body 94. The anti-slip portion 98 is formed around the central axis O5. The anti-slip portion 98 has a pair of intersecting ridge portions 98a and 98b formed at a boundary between an outer circumferential surface of the distal end portion 96C and an outer circumferential surface of the central portion 94B. The pair of intersecting ridge portions 98a and 98b are formed at a predetermined distance in the axial direction by a connection surface 99 that is present therebetween.

In the pair of intersecting ridge portions 98a and 98b, the intersecting ridge portion 98a on the distal end side A1 is configured of an intersecting ridge between the outer circumferential surface of the distal end portion 96C and the connection surface 99, and the intersecting ridge portion 98b on the proximal end side A2 is configured of an intersecting ridge between an outer circumferential surface of the central portion 94B and the connection surface 99. The pair of intersecting ridge portions 98a and 98b are located on the outer circumferential surfaces of the distal end portion 96C and the central portion 94B.

The connection surface 99 is an inclined surface that is inclined radially outward from the distal end side A1 to the proximal end side A2. In addition, the connection surface 99 is not limited to the inclined surface, but may be a curved surface having a concave or convex shape. A width of the connection surface 99 in the axial direction is not limited to the width shown in the drawing, and can be changed as appropriate.

In this embodiment, a boundary portion between the distal end portion 96C of which a diameter is narrowed toward the distal end side A1 and the central portion 94B which forms a body portion of the cup main body 94 with a maximum diameter is a portion of an outer circumferential surface of the forceps that presses against the biological tissue the most when the forceps is brought into contact with the biological tissue, and the anti-slip portion 98 is provided at this position. The pair of intersecting ridge portions 98a and 98b and the connection surface 99 all extend around the central axis O5, and the pair of intersecting ridge portions 98a and 98b are formed to be arranged at a predetermined interval in the axial direction via the connection surface 99. Therefore, axial slippage of the forceps 95 with respect to the biological tissue around the entire axis can be curbed.

### Fifth Embodiment

Next, a configuration of forceps pieces (a first forceps piece, a second forceps piece) of a fifth embodiment of the present disclosure will be described.

In the following description, the description of the same configuration as in the above embodiment will be omitted, and only the different parts will be described in detail.

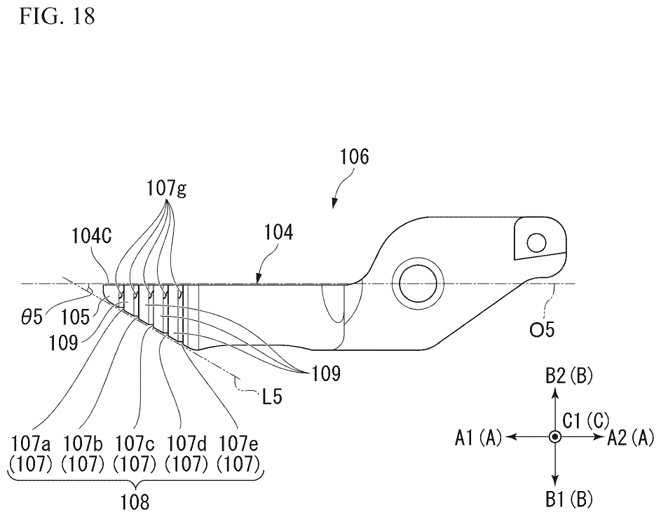

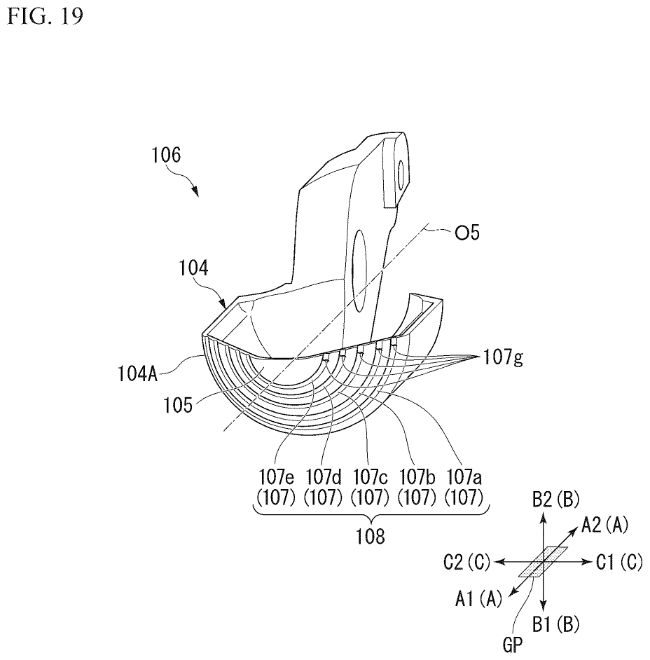

FIG. 18 is a side view showing a forceps piece 106 of the fifth embodiment. FIG. 19 is a perspective view showing the forceps piece 106 of the fifth embodiment.

As shown in FIG. 18, in the forceps piece 106 of the fifth embodiment, a shape of the distal end side A1 is different from that of the above-described embodiments.

A forceps is formed by combining the forceps piece 106 shown in FIG. 18 with another forceps piece having a similar shape in the up-down direction. Since the pair of forceps pieces constituting the forceps have the same configuration, only one forceps pieces 106 will be described in detail here.

The forceps piece 106 of this embodiment has an anti-slip portion 108 on an outer circumferential surface of a distal end portion 104C of a cup main body 104. The anti-slip portion 108 has a plurality of grooves 107 recessed radially inward from the outer circumferential surface of the distal end portion 104C. In this embodiment, there are five grooves 107a, 107b, 107c, 107d, and 107e, but the number of grooves 107 is not limited thereto and can be changed as appropriate. The five groove portions 107a, 107b, 107c, 107d, and 107e are formed at equal intervals from each other in the longitudinal direction A, but the present disclosure is not limited thereto, and the intervals between the groove portions 107 can be changed as appropriate and do not have to be equal.

Each of the grooves 107 has a semicircular arc shape that extends around the central axis O5, and when the forceps piece 106 is seen from the distal end side A1, the five grooves 107 are formed in a striped pattern. As shown in FIG. 18, an inner surface 107g of each of the grooves 107 faces radially outward and can be seen from the same direction. Also, each of the inner surfaces 107g faces not only radially outward but also toward the distal end side A1 as shown in FIG. 19 and can be seen from the same direction.

Four connection surfaces 109 are formed between the five grooves 107 arranged in the axial direction. Each of the connection surfaces 109 is inclined radially outward from the distal end side A1 in the longitudinal direction A to the proximal end side A2. Each of the connection surfaces 109 is inclined at a predetermined angle with respect to the central axis O5, and inclination angles are equal to each other. As shown in FIG. 18, each of the connection surfaces 109 is located on a virtual line L5 that is inclined at a predetermined angle θ5 with respect to the central axis O5.

As shown in FIG. 18, a width of the groove 107 in the longitudinal direction A is smaller than a width of the connection surface 109 in the longitudinal direction A. Also, as shown in FIG. 19, a width of the groove 107 in the radial direction is smaller than a width of the connection surface 109 in the radial direction.

According to the configuration of this embodiment, by forming the plurality of grooves 107 extending around the axis in the longitudinal direction A on the outer circumferential surface of the distal end portion 104C of the cup main body 104 of the forceps piece 106, the unevenness on the outer circumferential surface of the distal end portion 104C is increased, thereby further enhancing the anti-slip effect on biological tissue. Due to the anti-slip portion 108 having such a plurality of grooves 107, when the forceps piece 106 (the forceps) comes into contact with biological tissue, a part of the biological tissue enters each of the grooves 107, and when the forceps pieces 106 is about to start slipping, the biological tissue gets caught on the opening-side outer circumferential edges of the grooves 107 arranged in the axial direction, thereby preventing the forceps pieces 106 from sliding in the axial direction. In this manner, due to the non-slip portion 108 of the forceps piece 106, appropriate hooking to the biological tissue occurs, thereby making the forceps pieces 106 less likely to slip, and making it possible to stabilize the position of the forceps with respect to the biological tissue.

Further, the distal end portion 105 of the cup main body 104 has a smoothly curved surface and is not sharp.

Although the embodiments of the present disclosure have been described above, each configuration and combinations thereof in the embodiments are merely examples, and addition, omission, substitution, and other modifications of the configurations are possible without departing from the gist of the present disclosure. Furthermore, the present disclosure is not limited to the embodiments.

For example, in the above embodiment, the first forceps piece and the second forceps piece, which are arranged opposite each other to form the forceps, are configured to be able to open and close in directions that intersect the central axis toward the distal end side of the forceps, but the configuration of the forceps is not limited thereto. For example, one of the forceps pieces may be fixed, and only the other forceps piece may be opened and closed. Furthermore, one of the pair of forceps pieces may be replaced with a cover portion, and the forceps may be configured by combining the forceps piece and the cover portion. Furthermore, the fixed forceps piece and the cover portion may be integrally formed with the frame 31 of the support member 3.

The present disclosure can be applied to a biopsy forceps used for lung cancer examinations and the like, an endoscopic treatment system equipped with the biopsy forceps, and a method for collecting tissue using the endoscopic treatment system.

What is claimed is:

1. A biopsy forceps, comprising:
a first forceps piece having a first cup having a body portion, a distal end portion located distally relative to the body portion in a longitudinal direction of the first forceps piece and a first recess, the first recess having an opening; and
a second forceps piece facing the opening,
wherein the first forceps piece is configured to open and close in a first direction intersecting with the longitudinal direction of the first forceps piece relative to the second forceps piece,
an outer circumferential surface of the distal end portion has an inclined surface being flat and inclined so that a dimension of the distal end portion in one direction perpendicular to the longitudinal direction becomes smaller toward a distal end of the first forceps piece.

2. The biopsy forceps according to claim 1, wherein the outer circumferential surface of the distal end portion has a first inclined surface and a second inclined surface,
the first inclined surface is the inclined surface, and
the second inclined surface is flat and inclined relative to the longitudinal direction of the first forceps piece so that a width of the distal end portion in a second direction perpendicular to the longitudinal direction of the first forceps piece and the one direction gradually becomes smaller toward the distal end side.

3. The biopsy forceps according to claim 2, wherein the outer circumferential surface of the distal end portion has a ridge portion located between the first inclined surface and the second inclined surface, and
when seen from the distal end of the first forceps piece toward a proximal end of the first forceps piece, a distal end of the ridge portion is located radially inward of a proximal end of the ridge portion.

4. The biopsy forceps according to claim 1, wherein the second forceps piece has a second cup having a second recess, and
when the first forceps piece and the second forceps piece are in a closed state, the first recess and the second recess face each other.

5. The biopsy forceps according to claim 1, wherein the distal end portion has a distal end surface located a distal end of the distal end portion in the longitudinal direction of the first forceps piece, and
wherein the distal end surface is located proximally relative to an intersection point where a straight line intersects with an axis extending in the longitudinal direction, the straight line connecting a first point and a second point, the first point located between an outer circumferential surface of the body portion and the outer circumferential surface of the distal end portion, the second point is located between the outer circumferential surface of the distal end portion and the distal end surface,

6. The biopsy forceps according to claim 5, wherein when seen from the distal end of the first forceps piece toward the proximal end of the first forceps piece, the distal end surface has a rectangular shape.

7. The biopsy forceps according to claim 1, wherein the outer circumferential surface of the distal end portion has an anti-slip portion against the biological tissue.

8. The biopsy forceps according to claim 3, wherein the outer circumferential surface of the distal end portion has an anti-slip portion against the biological tissue, and
the anti-slip portion is configured of the ridge portion.

9. The biopsy forceps according to claim 7, wherein, in the inclined surfaces, the anti-slip portion is located on a position being farthest from the opening in the first direction.

10. The biopsy forceps according to claim 7, wherein the anti-slip portion is located on the inclined surface.

11. The biopsy forceps according to claim 10, wherein an inclination angle of the inclined surface relative to an axis extending in the longitudinal direction is within a range of 15° or more and 45° or less;

12. The biopsy forceps according to claim 7, wherein the outer circumferential surface of the distal end portion has a plurality of surfaces being flat or curved, and
the anti-slip portion is located between the plurality of surfaces.

13. The biopsy forceps according to claim 7, wherein the outer circumferential surface of the distal end portion has a plurality of surfaces being flat or curved, and
the anti-slip portion is located on at least one of the plurality of surfaces.

14. The biopsy forceps according to claim 7, wherein the anti-slip portion is located around an axis extending in the longitudinal direction along an ridge portion between the outer circumferential surface of the distal end portion and an outer circumferential surface of the body portion.

15. The biopsy forceps according to claim 1, wherein the body portion has a through hole communicating with the first recess.

16. The biopsy forceps according to claim 1, wherein a collected amount of the biological tissue being collectable by the first recess located is within a range of 0.7 mm³ or more and 6.0 mm³ or less.

17. The biopsy forceps according to claim 1, wherein when the first forceps piece and the second forceps piece are closed, a maximum outer diameter of the first forceps piece and the second forceps piece is 2.2 mm or less.

18. The biopsy forceps according to claim 1, wherein the outer circumferential surface of the distal end portion has an uneven portion configured to diffusely reflect ultrasonic waves.

19. An endoscopic treatment system comprising:
an endoscope configured to insert into a bronchus, and
the biopsy forceps according to claim 1.

20. A method for extracting tissue, comprising:
inserting a biopsy forceps in a closed state into an inside of a lumen, and
advancing the biopsy forceps to a vicinity of a puncture hole formed by puncturing a puncture needle from the inside of the lumen;
inserting the biopsy forceps into the puncture hole from a distal end side of the biopsy forceps; and
opening and closing the biopsy forceps inside the puncture hole to collect a part of the biological tissue.
